(19) United States
(12) Patent Application Publication
(10) Pub. No.: US 2008/0226738 A1
(43) Pub. Date: Sep. 18, 2008

(54) Sustained-Released Pellet Formulation of Alpha1-Receptor Antagonist and Process For the Preparation Thereof

(75) Inventors: Kwang Hyun Shin, Gyeonggi-do (KR); Young Hee Shin, Gyeonggi-do (KR); Sung Ah Bin, Gyeonggi-do (KR); Jung Ju Kim, Gyeonggi-do (KR)

Correspondence Address: SUGHRUE MION, PLLC, 2100 PENNSYLVANIA AVENUE, N.W., SUITE 800, WASHINGTON, DC, 20037, US

(73) Assignee: Amorepacific Corporation, Seoul (KR)

(21) Appl. No.: 12/064,174

(22) PCT Filed: Aug. 11, 2006
(86) PCT No.: PCT/KR06/03156

§ 371 Date: Apr. 9, 2008

(30) Foreign Application Priority Data
Aug. 19, 2005 (KR) 10-2005-0076449

(51) Int. Cl. A61K 9/52; A61P 35/00; A61P 43/00; A61P 13/08

(52) U.S. Cl. 424/494

(57) ABSTRACT

A sustained-release pellet formulation comprising: a pellet core comprising an α1-receptor antagonist, a pellet-forming substance and a pharmaceutically acceptable excipient and a coating layer comprising an enteric coating substance and a water-insoluble polymer, which is coated on said pellet core maintains a therapeutically effective drug level in the blood for a sufficient time without an initial burst and sustains the release of the drug even in the small intestine due to the water-insoluble polymer in the coating layer

### FIELD OF THE INVENTION

The present invention relates to a sustained-release pellet formulation of an α1-receptor antagonist for the treatment of urinary disorders associated with benign prostatic hyperplasia; and a method for preparing same.

### BACKGROUND OF THE INVENTION

Activation of the α₁ receptor causes the smooth muscle cells to contract, which brings such effects as constricting the vasculature, raising the blood pressure, and constricting the urinary tract to restricting the urine flow. Compounds which block the α₁ receptor exert the opposing effects.

Thus, α₁ receptor antagonists are potentially effective therapeutic agents for hypertension, congestive heart failure, and other cardiovascular diseases. Additionally, specific α₁-receptor antagonists such as tamsulosin, alfuzosin, doxazosin and terazosin have also been developed for the treatment of urinary symptoms suggestive of benign prostatic hyperplasia. By blocking α₁-receptors in the tissue such as prostate, the neck of bladder and urethra, α₁-receptor antagonists lead to the relaxation of smooth muscles of these organs to reduce causative obstruction. However, as α₁-receptors are also in the blood vessel, uncontrolled-release of α₁-receptor antagonists can cause vasodilatation or postural hypotension, which leads dizziness, orthostatic hypotension and syncope (See, Martin C. Michel, Eur Urol Supl., 4:15-24, 2005).

Therefore, it is very important to maintain a therapeutically acceptable level of α₁-receptor antagonists in the blood without sudden drug release in the early stage and various sustained-release pellet formulations have been developed for that purpose.

U.S. Pat. No. 4,772,475 disclosed that a pharmaceutical formulation having an excellent controlled-release characteristic can be obtained by adding a release controlling agent to a mixture of a physiologically active substance and unit-forming substance in an amount of at least 50% by weight based on the unit without enteric coating. However, the results in the test for release characteristics of the above formulation according to standardized Pharmacopoeial method (paddle, 150 rpm) show that the release ranged from 16.2 to 60.4% of the active compound in one hour in a simulated gastric fluid, and such a release rate is generally not sufficient for an extended-release dosage form.

Further, US Patent Pub. No. 2004/0096502 disclosed that a pellet formulation prepared by granulating a mixture of tamsulosin hydrochloride, microcrystalline cellulose, acrylic polymer and water and coating the pellet with an acid-resistant acrylic polymer (i.e. enteric coating substance) exhibits a dissolution release profile in which less than 10% of tamsulosin is released during the first two hours in a simulated gastric fluid (pH 1.2). However, as enteric coating substance quickly dissolves upon entering the small intestine, a satisfactory sustained-release profile of tamsulosin is not obtainable.

The present inventors have therefore endeavored to develop a pellet formulation that can maintain a therapeutically effective level of an α₁-receptor antagonist in the blood for a sufficiently long time without initial burst release of the drug, and have found that a pellet formulation comprising a controlled-release pellet core coated with a layer comprising an enteric coating substance and a water-insoluble polymer exhibits a satisfactory constant release profile of the drug, which was not achieved in the art.

### SUMMARY OF THE INVENTION

Accordingly, an object of the present invention is to provide a sustained-release pellet formulation of an α₁-receptor antagonist, which releases the drug continuously in the gastrointestinal tract without initial burst.

Other object of the present invention is to provide a method for preparing said formulation.

In accordance with one aspect of the present invention, there is provided a sustained-release pellet formulation comprising: a pellet core comprising an α₁-receptor antagonist, a pellet-forming susbstance and a pharmaceutically acceptable excipient and a coating layer comprising an enteric coating substance and a water-insoluble polymer, which is coated on said pellet core.

In accordance with another aspect of the present invention, there is provided a method for preparing a sustained release pellet formulation, which comprises:

(1) mixing an α₁-receptor antagonist, a pellet-forming substance and a pharmaceutically acceptable excipient, and granulating the resulting mixture by spraying thereto a binder solution, to obtain a pellet core; and

(2) coating the pellet core with a coating solution comprising an enteric coating substance and a water-insoluble polymer.

### BRIEF DESCRIPTION OF THE DRAWINGS

The above and other objects and features of the present invention will become apparent from the following description of the invention, when taken in conjunction with the accompanying drawing which shows:

FIG. 1: active ingredients dissolution profiles of the sustained-release pellet formulations prepared in Examples 4 and 5 and Comparative Example 1 of the present invention.

### DETAILED DESCRIPTION OF THE INVENTION

The components of the pellet formulation of the present invention are described in detail as follows:

### 1. Pellet Core

The pellet core comprises an α₁-receptor antagonist as an active ingredient, a pellet-forming substance and a pharmaceutically acceptable excipient, which plays the primary role in controlling the release of the drug. It has a diameter ranging from 0.2 to 2 mm, preferably, 0.5 to 1.5 mm. When the size of the pellet core is too small, it becomes difficult to control the drug release, and when too large, it becomes difficult to fill into a final unit dose with desired content homogeneity.

### 1) Pharmacologically Active Ingredient

In the present invention, α₁-receptor antagonists are used as active ingredients. Representative examples of the α₁-receptor antagonist include tamsulosin, alfuzosin, doxazosin, terazosin and a pharmaceutically acceptable salt thereof.

The active ingredient may be used in an amount of 0.05 to 20 wt %, preferably 0.1 to 10 wt % based on the total weight of the pellet formulation.

### 2) Pellet-forming Agent

Exemplary pellet-forming agents include microcrystalline cellulose, low-substituted hydroxypropylcellulose, chitin, chitosan and a mixture thereof. The pellet-forming agent may be used in an amount of 20 to 95 wt %, preferably 50 to 90 wt % based on the total weight of the pellet formulation.

When the amount of the pellet-forming agent is less than 20 wt %, the deviation of drug-release becomes greater due to poor sphericity and broad particle size distribution.

### 3) Pharmaceutically Acceptable Excipient

The pellet core of the present invention may further comprise at least one of the known pharmaceutically acceptable excipients such as a binder, a lubricant, a diluent, a disintergrant, an absorbent, a colorant, a flavouring agent, a sweetener and the like.

Exemplary binders include water, a mixture of water and ethanol, aqueous solution of a water-soluble polymer, and aqueous suspension, aqueous emulsion and organic solution of a water-insoluble polymer. Representative examples of the water-soluble polymer include hydroxypropylcellulose, hydroxypropylmethylcellulose, hydroxyethylcellulose, polyvinylpyrrolidone, copovidon, polyvinyl alcohol, and the like. Representative examples of the water-insoluble polymer include acrylic copolymers, for example, Eudragit™ L30D-55, Eudragit™ FS30D, Eudragit™ RL30D, Eudragit™ RS30D, Eudragit™ NE30D (Deggusa), Acryl-Eze™ (Colorcon Co.); polyvinylacetate, for example, Kollicoat™ SR 30D (BASF Co.); cellulose derivatives such as ethylcellulose, cellulose acetate, for example, Surelease™ (Colorcon Co.), Aquacoat™ ECD and Aquacoat™ CPD (FMC Co.).

Also, lubricants (for example: silica, talc, stearic acid, magnesium stearate, calcium stearate and/or polyethylene glycol), opacifiers (for example: titanium oxide), diluents (for example: lactose, dextrose, sucrose, mannitol, sorbitol, cellulose and/or glycine), disintergrants (for example: starch, agar, alginic acid and sodium salt), colorants and the like may be used as needed.

The pharmaceutically acceptable excipients may be used in an amount of 2 to 70 wt %, preferably 5 to 50 wt % based on the total weight of the pellet formulation.

### 2. Coating Layer

The coating layer comprises an enteric coating substance and a water-insoluble polymer to control the release of the drug secondly, and may be employed in an amount ranging from 1 to 20 wt %, preferably 2 to 15 wt % based on the total weight of the pellet formulation. A weight ratio of the enteric coating substance: the water-insoluble polymer in the coating layer ranges from 9:1 to 1:9 depending on the type of drug, but is not limited thereto.

### 1) Enteric Coating Substance

The enteric coating substance of the present invention is the enterosoluble substance that will dissolve at a pH above 5.0. Such enterosoluble substance includes methacrylate copolymer such as Eudragit™ L, S and FS30D (Deggusa Co.), hydroxypropylmethylcellulose phthalate, hydroxypropylmethylcellulose acetate succinate, cellulose acetate phthalate, and the like.

The plasticizers can be added to the enteric coating substance as needed in the range of 0 to 30 wt %. Exemplary plasticizers include polyethylene glycol, triethyl citrate, triacetin, triacetin citrate, castor oil, dibutylsebacate, dibutyltartrate, diethyl phthalate, glycerin and the like.

### 2) Water-insoluble Polymer

Exemplary water-insoluble polymers include acrylic copolymers, for example, Eudragit™ RL30D, Eudragit™ RS30D, Eudragit™ NE30D (Deggusa); polyvinylacetate, for example, Kollicoat™ SR 30D (BASF Co.); cellulose derivatives such as ethylcellulose, cellulose acetate, for example, Surelease™ (Colorcon Co.), Aquacoat™ ECD.

### 3) Pharmaceutically Acceptable Excipient

The above-described pharmaceutically acceptable excipients can be added to the coating layer of the present invention as needed.

Furthermore, the present inventive provides a method for preparing a sustained release pellet formulation, which comprises:

(1) mixing an α₁-receptor antagonist, a pellet-forming substance and a pharmaceutically acceptable excipient, and granulating the resulting mixture by spraying thereto a binder solution, to obtain a pellet core; and

(2) coating the pellet core with a coating solution comprising an enteric coating substance and a water-insoluble polymer.

In the present invention, the enteric coating substance and the water-insoluble polymer in the coating solution of the step 2 may be used in the form of aqueous suspension, aqueous emulsion or organic solution thereof or used directly.

Subsequently, the pellet formulation prepared can either be filled into capsules or be compressed into tablets with appropriate excipients

The following Examples are given for the purpose of illustration only, and are not intended to limit the scope of the invention.

### EXAMPLE 1

### Preparation of Sustained-release Pellet Formulations

### (1) Preparation of Pellets Comprising an Active Ingredient

1.0 g of tamsulosin hydrochloride (Ragactives, spain), 747 g of microcrystalline cellulose and 16 g of talc were thoroughly mixed in a centrifugal fluidized bed granulator (GPCG-1, Glatt, German) for about 1 minute. A binder solution (120 g of Eudragit™ L30D-55 in 580 g of water) was sprayed onto the mixture in the granulator to form pellets. The pellet-manufacturing conditions are listed in Table 1. The pellets thus obtained were spherical granules having diameters ranging from 0.5 to 1.4 mm as shown in Table 2.

TABLE 1

| | |
|---|---|
| Equipment Name | Fluid Bed System |
| Model Name | GPCG-1 |
| Spray Type | Tangential spray |

| | | |
|---|---|---|
| | Granulation | Drying |
| Temperature | Input: 30° C. | Input: 60~70° C. |
| | Output: 15~25° C. | Output: 35~40° C. |
| | Product: 15~25° C. | Product: 35~40° C. |

| | |
|---|---|
| Spray Speed | 10~30 ml/min |
| Spray Pressure | 1~2 bar |
| Rotation Speed | 300~700 rpm |

TABLE 2

| Particle Size (μm) | Weight (g) | Proportion (%) |
|---|---|---|
| >1400 | 0.45 | 0.45 |
| 710~1400 | 9.77 | 9.76 |
| 500~710 | 88.39 | 88.34 |
| 355~500 | 1.45 | 1.45 |
| <355 | — | — |
| Total | 100.06 | 100.00 |

(2) Coating the Pellets with a Drug Release Controlling Layer

The tamsulosin hydrochloride pellets obtained in step (1) (800 g) were coated with a drug release controlling layer having compositions listed in Table 3. The coating conditions are listed in Table 4. Finally, 168 mg of coated pellets each containing 0.2 mg of tamsulosin hydrochloride were obtained.

TABLE 3

| Composition | Weight (g) | Solids (g) |
|---|---|---|
| Eudragit ™ L30D-55 | 47.62 | 14.29 |
| Eudragit ™ NE30D | 47.62 | 14.29 |
| Triethylcitrate | 2.86 | 2.86 |
| Talc | 8.57 | 8.57 |
| Water | 116.67 | — |
| Total | 223.34 | 40.01 |

TABLE 4

| | |
|---|---|
| Equipment Name | Fluid Bed System |
| Model Name | GPCG-1 |
| Spray Type | Bottom spray |

| | | |
|---|---|---|
| | Coating | Drying |
| Temperature | Input: 40~45° C. | Input: 50~60° C. |
| | Output: 25~30° C. | Output: 35~40° C. |
| | Product: 25~30° C. | Product: 35~40° C. |

| | |
|---|---|
| Spray Speed | 5~10 ml/min |
| Spray Pressure | 1~2 bar |

### Example 2

### Preparation of Sustained-release Pellet Formulations

### (1) Preparation of Pellets Comprising an Active Ingredient

24.25 g of doxazosin mesylate (Cipla, India), 400 g of microcrystalline cellulose, 295.75 g of calcium phosphate dibasic and 40 g of talc were thoroughly mixed in a centrifugal fluidized bed granulator (GPCG-1, Glatt, German) for about 1 minute. A binder solution (133.3 g of Eudragit™ L30D-55 in 600 g of water) was sprayed onto the mixture in the granulator to form pellets. The pellet-manufacturing conditions are listed in Table 1. The pellets thus obtained were spherical granules having diameters ranging from 0.5 to 1.4 mm as shown in Table 5.

TABLE 5

| Particle Size (μm) | Weight (g) | Proportion (%) |
|---|---|---|
| >1400 | 8.78 | 8.79 |
| 710~1400 | 79.22 | 79.28 |
| 500~710 | 11.41 | 11.42 |
| 355~500 | 0.52 | 0.52 |
| <355 | — | — |
| Total | 99.93 | 100.01 |

(2) Coating the Pellets with a Drug Release Controlling Layer

The doxazosin mesylate pellets obtained in step (1) (700 g) were coated with a drug release controlling layer having compositions listed in Table 6. The coating conditions are listed in Table 4. Finally, 176 mg of coated pellets each containing 4.85 mg of doxazosin mesylate were obtained.

TABLE 6

| Composition | Weight (g) | Solids (g) |
|---|---|---|
| Eudragit ™ L30D-55 | 35 | 10.5 |
| Eudragit ™ NE30D | 81.67 | 24.5 |
| Triethylcitrate | 2.5 | 2.5 |
| Talc | 10.5 | 10.5 |
| Water | 110.33 | — |
| Total | 240 | 48 |

### Example 3

### Preparation of Sustained-release Pellet Formulations

### (1) Preparation of Pellets Comprising an Active Ingredient

50 g of alfuzosin hydrochloride (Heumann PCS, German), 550 g of microcrystalline cellulose, 120 g of calcium phosphate dibasic and 40 g of talc were thoroughly mixed in a centrifugal fluidized bed granulator (GPCG-1, Glatt, German) for about 1 minute. A binder solution (133.3 g of Eudragit™ L30D-55 in 600 g of water) was sprayed onto the mixture in the granulator to form pellets. The pellet-manufacturing conditions are listed in Table 1. The pellets thus obtained were spherical granules having diameters ranging from 0.7 to 1.4 mm as shown in Table 7.

TABLE 7

| Particle Size (μm) | Weight (g) | Proportion (%) |
|---|---|---|
| >1400 | 4.48 | 4.48 |
| 710~1400 | 91.96 | 92.04 |
| 500~710 | 3.24 | 3.24 |
| 355~500 | 0.23 | 0.23 |
| <355 | — | — |
| Total | 99.91 | 99.99 |

(2) Coating the Pellets with a Drug Release Controlling Layer

The alfuzosin hydrochloride pellets obtained in step (1) (800 g) were coated with a drug release controlling layer having compositions listed in Table 8. The coating conditions are listed in Table 4. Finally, 176 mg of coated pellets each containing 10 mg of alfuzosin hydrochloride were obtained.

TABLE 8

| Composition | Weight (g) | Solids (g) |
|---|---|---|
| Eudragit ™ L30D-55 | 16.67 | 5 |
| Eudragit ™ NE30D | 150 | 45 |
| Triethylcitrate | 5 | 5 |
| Talc | 15 | 15 |
| Water | 163.34 | — |
| Total | 350 | 70 |

### Examples 4 to 5

### Preparation of Sustained-release Pellet Formulations

### (1) Preparation of Pellets Comprising an Active Ingredient

After sufficiently mixing 0.5 g of tamsulosin hydrochloride and 351.5 g of microcrystalline cellulose, a binder solution (160 g of Eudragit™ L30D-55 in 230 g of water) was added to the mixture and the resultant mixture was granulated by a high speed mixer (NMG-5L, Nara, Japan) to form pellets. The pellets thus obtained were spherical granules having diameters ranging from 0.5 to 1.4 mm as shown in Table 9.

TABLE 9

| | Example 4 | | Example 5 | |
|---|---|---|---|---|
| | | Proportion | | Proportion |
| (Particle size (μm) | Weight (g) | (%) | Weight (g) | (%) |
| >1400 | 0.73 | 0.73 | 1.3 | 1.3 |
| 710~1400 | 16.72 | 16.72 | 34.04 | 34.05 |
| 500~710 | 82.24 | 82.22 | 64.37 | 64.4 |
| 355~500 | 0.33 | 0.33 | 0.25 | 0.25 |
| <355 | — | — | — | — |
| Total | 100.02 | 100.00 | 99.96 | 100.00 |

(2) Coating the Pellets with a Drug Release Controlling Layer

The tamsulosin hydrochloride pellets obtained in step (1) (800 g) were coated with a drug release controlling layer having compositions listed in Table 10. The coating conditions are listed in Table 4. Finally, 168 mg of coated pellets each containing 0.2 mg of tamsulosin hydrochloride were obtained.

TABLE 10

| | Example 4 | | Example 5 | |
|---|---|---|---|---|
| Composition | Weight (g) | Solids (g) | Weight (g) | Solids (g) |
| Eudragit ™ | 57.13 | 17.14 | 38.1 | 11.43 |
| L30D-55 | | | | |
| Eudragit ™ | 38.1 | 11.43 | 57.13 | 17.14 |
| NE30D | | | | |
| Triethylcitrate | 2.86 | 2.86 | 2.86 | 2.86 |
| Talc | 8.57 | 8.57 | 8.57 | 8.57 |
| Water | 116.67 | — | 116.67 | — |
| Total | 223.33 | 40 | 223.33 | 40 |

### Comparative Example 1

### (1) Preparation of Pellets Comprising an Active Ingredient

After sufficiently mixing 0.5 g of tamsulosin hydrochloride and 351.5g of microcrystalline cellulose, a binder solution (160 g of Eudragit™ L30D-55 in 215 g of water) was added to the mixture and the resultant mixture was granulated by a high speed mixer (NMG-5L, Nara, Japan) to form pellets. The pellets thus obtained were spherical granules having diameters ranging from 0.3 to 0.7 mm as shown in Table 11.

TABLE 11

| Particle Size (μm) | Weight (g) | Proportion (%) |
|---|---|---|
| >1400 | 1.57 | 1.57 |
| 710~1400 | 7.78 | 7.78 |
| 500~710 | 10.05 | 10.05 |
| 355~500 | 78.01 | 78.03 |
| <355 | 2.57 | 2.57 |
| Total | 99.98 | 100.00 |

(2) Coating the Pellets with a Drug Release Controlling Layer

The tamsulosin hydrochloride pellets obtained in step (1) (800 g) were coated with a drug release controlling layer having compositions listed in Table 12. The coating conditions are listed in Table 4. Coating was performed only with an enteric coating substance (Eudragit™ L30D-55) without any water-insoluble polymer. Finally, 168 mg of coated pellets each containing 0.2 mg of tamsulosin hydrochloride were obtained.

TABLE 12

| Composition | Weight (g) | Solids (g) |
|---|---|---|
| Eudragit ™ L30D-55 | 190.46 | 57.14 |
| Eudragit ™ NE30D | — | — |
| Triethylcitrate | 5.72 | 5.72 |
| Talc | 17.14 | 17.14 |
| Water | 233.34 | — |
| Total | 446.66 | 80 |

### Test Example 5

### Dissolution Test

For an amount corresponding to 0.2 mg of pellets of tamsulosin hydrochloride, the pellets prepared in Examples 4 and 5 and Comparative Example 1 were each filled into capsules and used as test samples. A dissolution test was conducted in accordance with the dissolution test method (2nd method) described in Korean Pharmacopeia at a rotation speed of 100 rpm by employing a mixture of 500 ml of No.1 liquid of disintegration test (pH 1.2) and 1 ml of reconstituted solution of polysorbate 80 (3→200) as a test liquid. 2 hours after the initiation of the test, 10 ml aliquot of eluate (1^{st} aliquot) was collected, and the test liquid was removed and 500 ml of phosphate buffer (pH 7.2, 37±0.5° C.) was added hereto. One hour thereafter, 10 ml aliquot of the eluate (2^{nd} aliquot) was collected, and 10 ml of fresh phosphate buffer was added thereto. Then, 10 ml aliquot of the eluate (3^{rd} aliquot) was collected 5 hrs after the initiation of the test. The 2^{nd} and 3^{rd} aliquots were each treated with 1.0 ml of 0.5 N hydrochloride. The tamsulosin hydrochloride concentration was determined by HPLC (High Performance Liquid Chromatography) for each of the above aliquots under the following conditions. Six samples for each aliquot were tested respectively.

Column: LUNA C18 (4.6×150 mm, 5 μm)

Detector: UV 225 nm

Flow rate: regulated so that the retention time for tamsulosin becomes about 6 minutes.

Injection Volume: 100 μl

Column Temperature: 40° C.

Mobile phase: The pH of a solution of perchloric acid (8.7 ml) and sodium hydroxide (3.0 g) in water (1900 ml) was adjusted to 2.0 with sodium hydroxide, water was added thereto to a final volume of 2000 ml, and then, 1400 ml of the solution was taken to be combined with 600 ml of actonitrile.

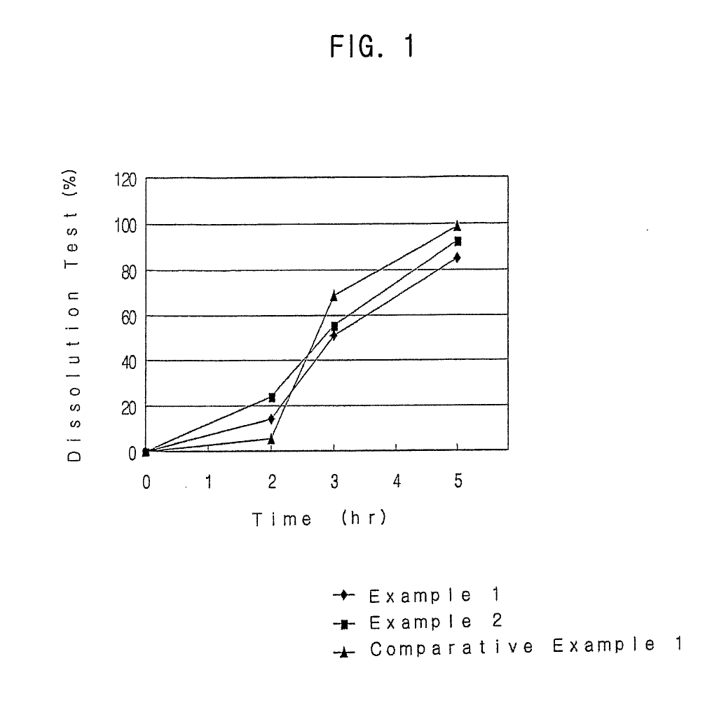

The results thus obtained are shown in Table 13 and FIG. 1.

TABLE 13

| | Dissolution rate (%) | | |
|---|---|---|---|
| Time (h) | Example 4 | Example 5 | Comparative Example 1 |
| 2 | 14.2 | 23.9 | 5.6 |
| 3 | 50.8 | 55.4 | 68.7 |
| 5 | 85 | 92.4 | 98.6 |

As shown in Table 13, the coated pellets of Examples 4 and 5 were capable of sustaining the release of the tamsulosin hydrochloride throughout the early stage at pH 1.2 and by later stage at pH 7.2. The coated pellet of Comparative Example 1 which does not contain the insoluble polymer in its coating layer could sustain the release of tamsulosin hydrochloride at pH 1.2, but showed a burst release behavior at the later stage of pH 7.2.

Above results show that the inventive pellet formulation (Examples 4 and 5) represses the initial burst release of the drug in the stomach owing to its enteric coating substance in its coating layer. Although the formulation loses the enteric coating substance when it enters the small intestine, it can sustain a satisfactory drug release due to the presence of a water-insoluble polymer in its coating layer.

While the embodiments of the subject invention have been described and illustrated, it is obvious that various changes and modifications can be made therein without departing from the spirit of the present invention which should be limited only by the scope of the appended claims.

What is claimed is:

1. A sustained-release pellet formulation comprising: a pellet core comprising an α₁-receptor antagonist, a pellet-forming susbstance and a pharmaceutically acceptable excipient and a coating layer comprising an enteric coating substance and a water-insoluble polymer, which is coated on said pellet core.

2. The sustained-release pellet formulation of claim 1, wherein the α₁-receptor antagonist is selected from the group consisting of tamsulosin, alfuzosin, doxazosin, terazosin and a pharmaceutically acceptable salt thereof.

3. The sustained-release pellet formulation of claim 1, wherein the pellet-forming substance is selected from the group consisting of microcrystalline cellulose, low-substituted hydroxypropylcellulose, chitin, chitosan and a mixture thereof.

4. The sustained-release pellet formulation of claim 1, wherein the amount of the pellet-forming substance ranges from 20 to 95% by weight based on the total weight of the pellet formulation.

5. The sustained-release pellet formulation of claim 1, wherein the amount of the coating layer ranges from 1 to 20% by weight based on the total weight of the pellet core.

6. The sustained-release pellet formulation of claim 1, wherein the weight ratio of the enteric coating substance and the water-insoluble polymer in the coating layer ranges from 9:1 to 1:9.

7. The sustained-release pellet formulation of claim 1, wherein the pellet core has a diameter ranging from 0.2 to 2.0 mm.

8. The sustained-release pellet formulation of claim 1, wherein the pharmaceutically acceptable excipient is selected from the group consisting of a binder and a lubricant.

9. The sustained-release pellet formulation of claim 8, wherein the binder is selected from the group consisting of water, a mixture of water and ethanol, an aqueous solution of a water-soluble polymer, and an aqueous suspension, aqueous emulsion and water-containing organic solvent solution of a water-insoluble polymer.

10. The sustained-release pellet formulation of claim 9, wherein the water-insoluble polymer is selected from the group consisting of acrylic copolymers, polyvinylacetate and cellulose derivatives.

11. The sustained-release pellet formulation of claim 1, wherein the enteric coating substance is an enterosoluble polymer which dissolves at a pH above 5.

12. The sustained-release pellet formulation of claim 11, wherein the enteric coating substance is selected from the group consisting of methacrylate copolymer, hydroxypropylmethylcellulose phthalate, hydroxypropylmethylcellulose acetate succinate and cellulose acetate phthalate.

13. The sustained-release pellet formulation of claim 1, wherein the water-insoluble polymer is selected from the group consisting of acrylic copolymers, polyvinylacetate and cellulose derivatives.

14. The sustained-release pellet formulation of claim 1, which is in the form of a capsule or a tablet.

15. A method for preparing a sustained release pellet formulation, which comprises: (1) mixing an α₁-receptor antagonist, a pellet-forming substance and a pharmaceutically acceptable excipient, and granulating the resulting mixture by spraying thereto a binder solution, to obtain a pellet core; and
(2) coating the pellet core with a coating solution comprising an enteric coating substance and a water-insoluble polymer.
